(19) United States
(12) Patent Application Publication
(10) Pub. No.: US 2024/0026289 A1
(43) Pub. Date: Jan. 25, 2024

(54) OSMOTICALLY-ACTIVE CLOSED-CELL COMPOSITE, OSMOTICALLY-ACTIVATED ACTUATOR AND ACTUATION METHOD

(71) Applicant: The Board of Trustees of the University of Illinois, Urbana, IL (US)

(72) Inventors: Shelby B. HUTCHENS, Champaign, IL (US); Amrita KATARUKA, Mountain View, CA (US)

(21) Appl. No.: 18/224,662

(22) Filed: Jul. 21, 2023

Related U.S. Application Data
(60) Provisional application No. 63/391,485, filed on Jul. 22, 2022.

(51) Int. Cl. C12N 5/04

(52) U.S. Cl. CPC C12N 5/04

(57) ABSTRACT

An osmotically-active closed-cell composite comprises a closed-cell structure including fluid-filled cells separated by cell walls, where the fluid-filled cells comprise water and a solute, and the cell walls comprise a polymer permeable to water and impermeable to the solute. The closed-cell structure is configured to undergo osmotically-induced swelling during exposure to an aqueous environment having a different chemical potential from the fluid-filled cells.

### RELATED APPLICATIONS

The present patent document claims the benefit of priority under 35 U.S.C. 119(e) to U.S. Provisional Patent Application 63/391,485, which was filed on Jul. 22, 2022, and is hereby incorporated by reference in its entirety.

### FEDERALLY SPONSORED RESEARCH OR DEVELOPMENT

This invention was made with government support under 1653676 awarded by the National Science Foundation. The government has certain rights in the invention.

### TECHNICAL FIELD

The present disclosure is related generally to porous polymer composites and more particularly to osmotically-active closed-cell composites.

### BACKGROUND

Plant tissues achieve striking, forceful movement driven by fluid flow rather than muscle. Often it is the tissue's ability to maintain an internal hydrostatic pressure above atmospheric levels, also known as turgor pressure, that enables the strength of these motions. By these means, plants resist gravity, penetrate soil, and overcome energetic barriers to leverage instability. Maintaining turgor pressure at equilibrium requires closed-cells to encapsulate the fluid. Because these encapsulating cell walls are solid, flow must occur via solution into and then diffusion through the wall material rather than proceeding via open pores between chambers as in typical poroelastic solids. Positive turgor pressure in the tissue relative to the environment creates a driving force for fluid to leave the tissue, leading to shrinkage. In contrast, osmotic pressure differences between the cell cavities and surrounding fluid draw fluid in, leading to swelling-induced deformation.

Plant-like features and functions have inspired innumerable device and material mimics. Hydrogels in particular are a well-known class of synthetic materials whose equilibrium swelling shares several features of this chemical-energy-driven mechanism. As such, they provide a useful reference against which to compare the swelling behavior of the plant tissue analogs (PTAs) described here. Similarities between hydrogels and PTAs include their high water content, generally >80% water by volume; internal chemical energy supply; and self-driven deformation in aqueous environments. These features have contributed to extensive recent interest in hydrogel materials as autonomous valves, soft robots, and mechanically active wound dressings, to name a few examples. Although such hydrogels have been made mechanically robust through recent double-network approaches, there remains a fundamental shortcoming inherent to their operation. Their modulus decreases, i.e., they soften, upon swelling due to the accompanying decrease in crosslink density as the polymer network expands to accommodate volume change. This feature limits hydrogel use when deformation is resisted by high force.

### BRIEF DESCRIPTION OF THE DRAWINGS

FIG. 1 provides a schematic illustration of an osmotically-active closed-cell composite, or plant tissue analog (PTA), and its swelling behavior in an aqueous environment having a different chemical potential than the fluid-filled cells.

FIG. 2A shows a scanning electron micrograph (SEM) image of dried plant parenchyma (apple tissue) showing densely packed, closed-cells or cavities of about 100 μm in size.

FIG. 2B shows an SEM image of a cross section of a representative PTA having analogous structure to the apple tissue of FIG. 2A.

FIG. 3 is a schematic of an osmotically-activated actuator including an osmotically-active closed-cell-composite attached to an object (e.g., a polymer layer) for actuation; the closed-cell composite can generate sufficient force upon swelling to bend the polymer layer and lift a 5-g weight.

FIG. 4 shows via schematics the actuation behavior of bilayer actuator comprising an osmotically-active closed-cell composite coupled to a polydimethylsiloxane (PDMS) layer.

FIG. 5 shows via schematics the actuation behavior of a bilayer actuator comprising a hydrogel coupled to a PDMS layer, where actuation fails with a 5-g load (bottom-most image).

FIG. 6A shows that osmotic potential drives water into the fluid-filled cells of the closed-cell composite, while infinite flow is resisted by the emergence of hydrostatic pressure; the graph illustrates the interplay of these energetic potentials for fluid flow: osmotic (solid line) and hydrostatic (heavy and light dashed lines), as the volume of the fluid-filled cells increases. The left hand side inset shows a traditional U-tube experiment where hydrostatic pressure is linear due to the pull of gravity on the difference in height between the pure and osmolyte phases, as shown by schematics adjacent to the y-axis; in contrast, with PTAs, turgor pressure (Pₜ) is nonlinear due to material nonlinearity accompanying the increase in wall stress for expanding cells, as shown by schematics adjacent to the x-axis.

FIG. 6B shows a schematic illustrating the effect of initial osmolyte (salt in this example) concentration and crosslink density on equilibrium swelling volume, indicated by the intersection of the curved lines. Dashed and bold lines of increasing saturation (labeled higher and lower salt concentration) denote osmotic potential for increasing initial salt concentration. The dashed lines of increasing saturation (labeled higher and lower crosslink density) represent turgor pressure Pₜ for increasing crosslinker (higher modulus, less stretchability).

FIG. 6C shows the swelling response of PTAs using a constant PDMS formulation (1A:1B) for the cell walls and varying initial salt concentration. Points and lines correspond to experimental data and exponential fits, respectively. PTAs with lower salt concentrations reach equilibrium (solid lines), while higher salt concentrations rupture (dashed lines).

FIG. 6D shows the swelling response of PTAs having constant initial salt concentration (0.01 m) and a varying PDMS formulation for the cell walls. Equilibrium swelling is inversely related to the amount of crosslinker in the PDMS.

FIG. 7A shows experimental equilibrium volume expansion ratio V_{eq}/V₀ of PTAs with varying initial salt loads (squares) and cell wall formulation (dashes). Position on the y-axis is determined by the intersection of the |Ψ_{Π,0}| curve (dashed lines) for the C₀ used and the corresponding V_{eq}/V₀. Solid and dotted lines represent the turgor pressure of Gent and neo-Hookean constitutive models, respectively.

FIG. 7B shows the composite Young's modulus of PTAs at zero turgor pressure as a function of cell wall modulus. Fit lines for Warner's and Nilsson's predictions use wall thickness and cell sizes of t=4 μm and r₀=50 μm, respectively.

FIG. 7C shows composite Young's modulus of swollen PTAs as a function of turgor pressure. Modulus is maintained or slightly increases with turgor pressure. The line at the top provides the dependence predicted by Nilsson et al. for comparison.

FIG. 8 shows that PTAs exhibit an intermediate response between plant tissue and hydrogels by plotting composite moduli versus swelling response of selected plant tissues (apple, carrot, potato, tomato), PTA, and hydrogels; to facilitate comparison of deformation-induced stiffening, the modulus E_{c} is normalized by its initial value.

FIG. 9 shows a soil dislodgement test to demonstrate actuation within confined environments, where the results reveal that the PTA dislodges enough sand to occupy more than twice its originally visible area, A/A₀>2. The images of the samples through the beaker bottom show the initial areas of the PTA and the hydrogel, and the areas after 72 h of swelling; the swollen sample extends outside of the black marker line, as indicated by the white dashed line.

### DETAILED DESCRIPTION

Synthetic hydrated materials share their characteristic stiffness and composition with biological tissues, which makes them promising biomaterial candidates. Their usefulness is further enhanced by their ability to actuate and mechanically respond to environmental stimuli. However, as indicated above, typical hydrated materials (hydrogels) lack the ability to apply forces large enough to support or functionally load tissues and organs. Inspired by plants' ability to structurally support and move themselves using osmosis-driven water pressure, synthetic plant tissue analogs (PTAs) demonstrated here represent a new class of hydrated soft composite capable of forceful, active motion. An encapsulated osmolyte means the material requires no external power source for operation, only a hydrated environment. When immersed in water, these PTAs—or osmotically-active closed cell composites—may reach a state of equilibrium governed by the initial osmolyte concentration (e.g., a higher concentration produces more swelling) and cell wall mechanical response (e.g., a stiffer cell wall yields less swelling). Given these behaviors, PTAs represent an alternate class of aqueous, autonomous synthetic materials that may be exploited as actuators in biomedical and other applications. Throughout this disclosure, the term “plant tissue analog” (or PTA) and “osmotically-active closed-cell composite” may be used interchangeably.

Referring to FIG. 1, the osmotically-active closed-cell composite 100 has a closed-cell structure 106 including cell walls 102 separating fluid-filled cells 104. The cell walls 102, which may be described as a continuous phase of the composite 100, comprise a polymer, and the fluid-filled cells 104 comprise water and a solute. The polymer is permeable to the water and impermeable, or substantially impermeable, to the solute. More specifically, the permeability of the polymer to the water may be at least three orders of magnitude higher than the permeability of the polymer to the solute. Consequently, when exposed to an aqueous environment 108 having a different chemical potential from the fluid-filled cells 104, water from the aqueous environment 108 diffuses through the cell walls 102 and into the fluid-filled cells 104, such that the closed-cell structure 106 undergoes osmotically-induced swelling, as illustrated in FIG. 1. The swelling may continue until chemical equilibrium is reached, at which point the chemical potential of the aqueous environment 108 and the fluid-filled cells 104 is the same. As discussed further below, a final swelling ratio V/V₀ of the closed-cell structure 106 and the rate at which swelling occurs may depend on one or more of the following: thickness of the cell walls, size of the fluid-filled cells, concentration of the solute in the water, constitutive response of the polymer (determined by the polymer network mechanical properties and microstructure, e.g., crosslink density), and/or chemical composition of the cell walls (affecting permeability to water).

The polymer of the cell walls 102 may comprise a homopolymer or a copolymer. Advantageously, the polymer may comprise an elastomer, that is, a natural or synthetic polymer having elastic or rubber-like properties. For example, the elastomer may include silicone, natural rubber, polybutadiene, styrene butadiene rubber, polyurethane, polyisoprene, and/or neoprene. In some examples, the cell walls may further include silica nanoparticles (e.g., sub-100 nm size particles), which may act as an emulsion stabilizer during fabrication and may serve to increase the stiffness of the cell walls compared to the polymer without silica reinforcements. In some examples, the silica nanoparticle concentration may be in a range from about 1 wt. % to about 10 wt. %. The cell walls 102 may have a thickness t of at least about 0.1 micron, or at least about 1 micron, and/or up to about 10 microns, or up to about 25 microns. A large wall thickness and/or the presence of a high concentration of silica nanoparticles in the cell walls 102 may be associated with a reduced final swelling ratio V/V₀ of the closed-cell structure 106.

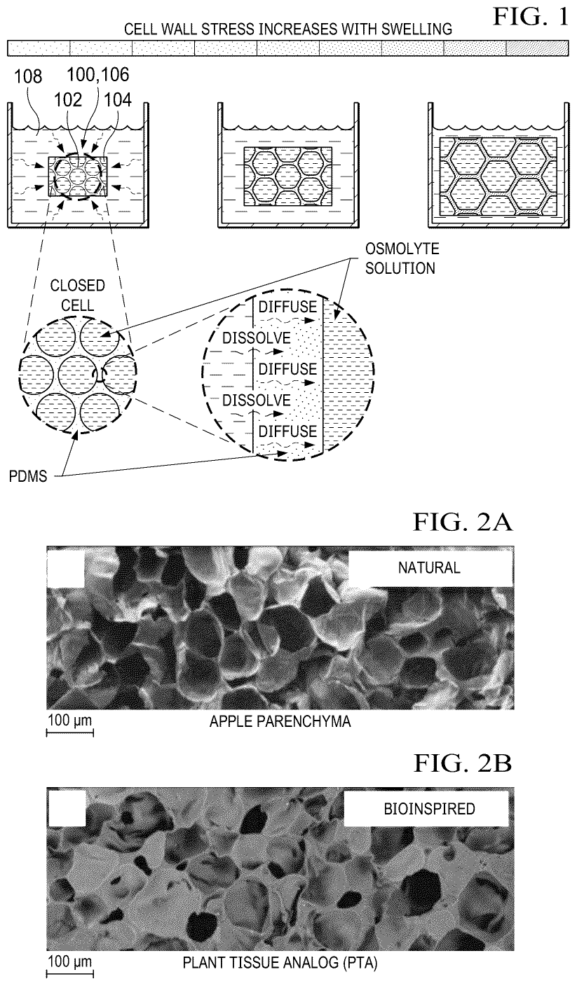

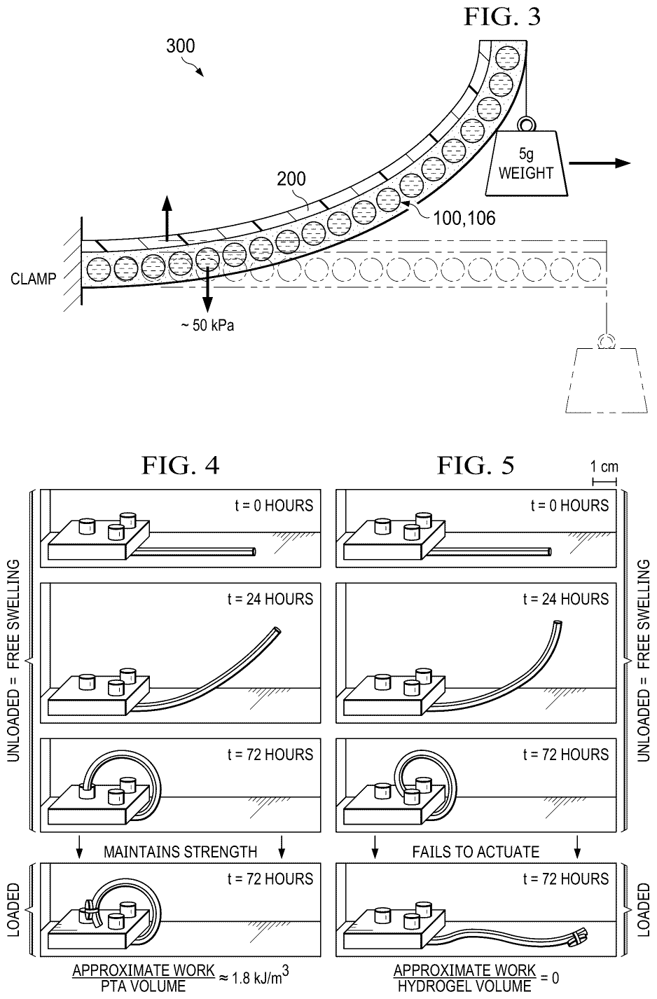

The fluid-filled cells 104 may be polyhedral or curved in shape. While the schematic of FIG. 1 illustrates a highly regular closed-cell structure 106 with hexagonal cells 104, scanning electron microscope (SEM) images of fabricated PTAs show that the cells may exhibit some cell-to-cell variation in size and shape, e.g., see FIG. 2B. For comparison, an SEM image of dried plant parenchyma (apple tissue) is shown in FIG. 2A. Typically, the fluid-filled cells 104 of the osmotically-active closed-cell composite 100 have a nominal or average cell size r₀ in a range from about 10 to about 500 microns. For example, the average cell size r₀ may be at least about 10 microns, at least about 25 microns, or at least about 50 microns, and/or the average cell size r₀ is typically no greater than about 500 microns, no greater than about 250 microns, or no greater than about 100 microns. The fluid-filled cells 104 may account for at least about 74 vol. % or at least about 80 vol. % of the closed-cell structure 106.

The solute contained in the fluid-filled cells 104 may comprise an osmolyte, which may be an organic osmolyte comprising a protein, an amino acid, a polyol, a sugar, a polysaccharide such as alginate, a methylamine, and/or urea. Also or alternatively, the solute may comprise an ionic compound such as sodium chloride (NaCl), potassium chloride, sodium phosphate, sodium bicarbonate, ammonium sulfate, sodium sulfate, ammonium acetate, or another salt. In some examples, the solute may comprise a hydrogel, such as hydrogel beads or particles suspended in the water; such a hydrogel suspension may be formed from hydrogel precursors that undergo crosslinking during or after curing of the cell walls 102. Accordingly, the fluid-filled cells may contain, in addition to a liquid (e.g., water), a gel that may function as an osmolyte. The fluid-filled cells are devoid or substantially devoid of any gases.

The solute may be dissolved and/or suspended in the water. In one example, the solute may be dissolved in the water at a concentration in a range from greater than zero to a saturation concentration. For example, in the case of NaCl, the solute may have a concentration in the range from about 0.01 m to about 0.2 m, wherein m denotes molality, the number of moles of the solute per mass (in kg) of the water. Notably, in contrast to prior work, the closed-cell structure 106 does not include glycerol, a solute found to be capable of diffusing through the cell walls 102. Other solutes or osmolytes, such as alginate, do not appreciably diffuse through the cell walls 102 and may enable a high viscosity of the liquid, which can eliminate the need for a centrifugation step during manufacturing. As indicated above, the closed-cell structure 106 is designed to be selectively permeable, that is, permeable to water but impermeable to the solute contained with the fluid-filled cells 104, such that the solute remains in the fluid-filled cells during swelling. More specifically, the permeability of the polymer to the water may be at least three orders of magnitude higher than the permeability of the polymer to the solute so as to maximize swelling and actuation. In examples where the fluid-filled cells may contain more than one solute, the cell walls 102 are impermeable to each of the solutes.

Experimental results described below show that these osmotically-active closed-cell composites or PTAs 100 may behave as non-vascular plant tissue. The osmotic pressure differential established by the osmolyte drives water into the fluid-filled cells 104, stretching the cell walls 102 and giving rise to turgor pressure, Pₜ, which provides a potential for counter-flow, eventually bringing the composite 100 into chemical equilibrium. Modifications of the closed-cell structure 106 (e.g., thickness of the cell walls 102, size of the fluid-filled cells 104, constitutive response of the polymer) may increase the stiffness (or elastic modulus E_{c}) of the closed-cell composites 100. In addition, the elastic modulus E_{c} may remain substantially constant or may increase during osmotically-induced swelling. Accordingly, the osmotically-active closed-cell composite 100 can apply significant actuation forces during swelling.

As illustrated in the schematic of FIG. 3, the closed-cell composite 100 may be in contact with, attached to or integrally formed with an object 200 to be actuated (e.g., moved and/or controlled), so as to form an osmotically-active actuator 300 suitable for biomedical, industrial or other applications. The object 200 may be an inanimate object, human tissue, and/or animal tissue. In one example, analogous to casts which hold bones in place while they heal, the osmotically-activated actuator 300 may be effective for holding tissues in place during healing. Beneficially, the osmotic actuator 300 could help to induce tissue-specific remodeling that facilitates regaining of tissue mechanical function via slow but dynamic stretching, in contrast to current technologies where wounds and/or injuries remain static and/or compressively loaded after therapy application or during healing. The object 200 to be actuated may be attached to the osmotically-active closed-cell composite 100 by an adhesive bond, or, as mentioned above, the object 200 may be integrally formed with the osmotically-active closed-cell composite 100, e.g., during a molding or printing process.

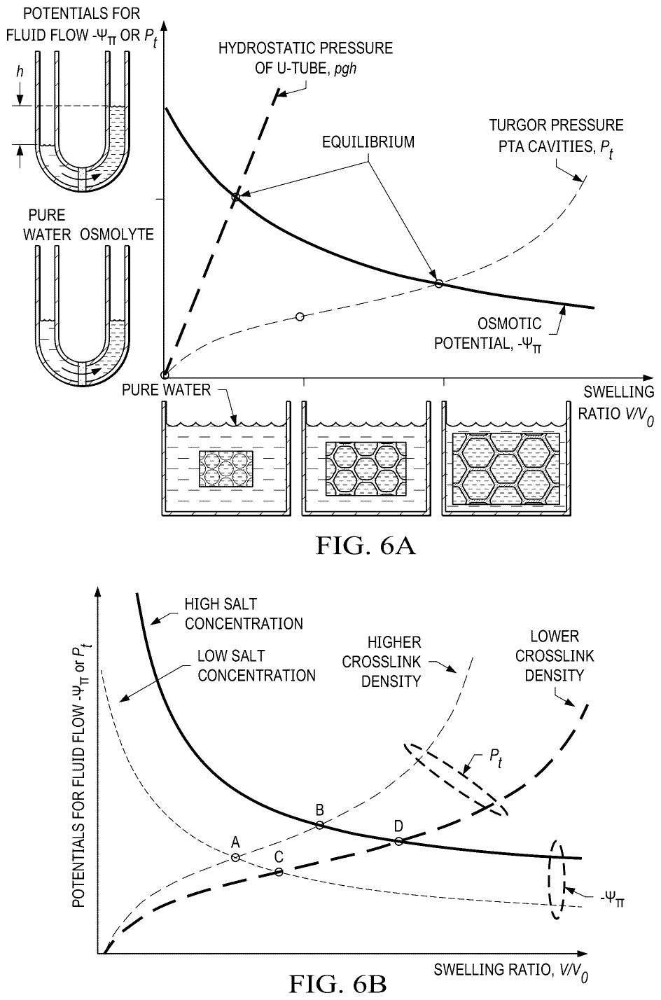

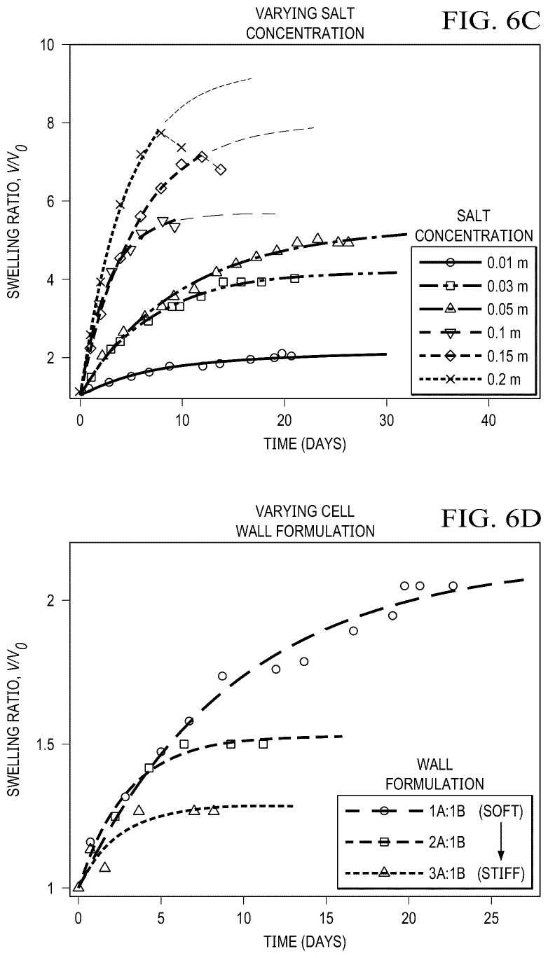

Accordingly, an osmotic actuation method may comprise providing an actuator 300 comprising an osmotically-active closed-cell composite 100 in contact with, attached to or integrally formed with an object 200 to be actuated, where the object 200 may be an inanimate object, human tissue, and/or animal tissue. As described above in reference to FIG. 1, the osmotically-active closed-cell composite 100 comprises a closed-cell structure 106 including cell walls 102 separating fluid-filled cells 104, where the cell walls 102 comprise a polymer, and the fluid-filled cells 104 comprise water and a solute. Importantly, the polymer is permeable to the water and substantially impermeable to the solute. The actuator 300 is exposed to an aqueous environment 108 having a different chemical potential from the fluid-filled cells 104 of the closed-cell structure 106. Due to the difference in chemical potential, water from the aqueous environment 108 diffuses through the cell walls 102 and enters the fluid-filled cells 104, and the closed-cell structure 106 undergoes osmotically-induced swelling. Consequently, a swelling force is applied to the object 200, and actuation is effected. The swelling of the closed-cell composite 100 may continue until chemical equilibrium is reached, that is, until the chemical potential of the fluid-filled cells matches that of the aqueous environment. The swelling force may be attributed to or aided by the constant or increasing stiffness (elastic modulus E_{c}) of the closed-cell structure 106 during osmotically induced swelling, which is analogous to the behavior of plants and is unattainable with hydrogels alone. Referring for example to the images of FIGS. 5 and 6, respectively, the actuation behavior of an actuator comprising an osmotically-active closed-cell composite coupled to a polydimethylsiloxane (PDMS) layer may be compared to that of an actuator comprising a hydrogel coupled to a PDMS layer. As shown in FIG. 5, swelling of the closed-cell composite induced by the water exposure leads, over time, to curling of the PDMS layer, even with an attached 5 g load. In contrast, when the hydrogel is coupled to the PDMS layer, as in FIG. 6, actuation fails with the 5 g load. These experiments are described in greater detail below.

Since the final swelling ratio V/V₀ of the closed-cell structure 106 may depend on thickness of the cell walls, size of the fluid-filled cells, concentration of the solute in the water, constitutive response of the polymer, and/or chemical composition of the cell walls, it may be possible to control the swelling, and consequently the actuation, by proper selection of these parameters.

The aqueous environment 108 to which the actuator 300 is exposed may comprise liquid water and/or water vapor. The exposure may entail submerging the actuator in the liquid water and/or water vapor; positioning the actuator in a flow path of the liquid water and/or water vapor; and/or spraying the actuator with the liquid water and/or water vapor.

The plant-like behavior of the osmotically-active closed-cell composite 100 is enabled by a fabrication technique—emulsion templating—that yields closed-cells with thin, selectively permeable cell walls and a readily varied fluid phase. Silicone or another elastomer is advantageously employed for the cell walls due to its inherent combination of high stretchability and selective permeability. The emulsion templating process may include (1) creation of a low-volume-fraction Pickering emulsion followed by centrifugation to a high-volume-fraction emulsion using components having different viscosities, (2) creation of a high-volume-fraction Pickering emulsion using components having substantially similar viscosities, and/or (3) fractionation to minimize or eliminate cell size gradients and curing/crosslinking to produce a solid closed-cell structure that closely resembles the structure of plant parenchyma tissue, as shown in FIGS. 2A and 2B. Curing or crosslinking may take place at room temperature (e.g., 20-25° C.), at an elevated temperature, and/or via exposure to light (e.g., ultraviolet (UV) light) and/or a chemical curing agent. Within the fluid-filled cells, a readily tunable osmolyte concentration serves to establish an osmotic driving force between the closed-cell composite and an aqueous environment, as illustrated in FIG. 1. To form a closed-cell composite 100 of a predetermined shape or to form an osmotically-activated actuator 300, prior to curing, the emulsion may be poured into a mold having a desired shape. The closed-cell composite 100 or actuator 300 may also or alternatively be cut into a desired shape after curing. An object 200 to be actuated may be integrally formed with the closed-cell composite 100 during curing, that is, a diffusion bond between the object 200 and the composite 100 may be obtained. Alternatively, an object 200 to be actuated may be attached to the closed-cell composite 100 after fabrication by an adhesive bond.

Swelling Response

As FIG. 6A illustrates, equilibrium swelling is associated with a balance between the osmotic driving force provided by the salt and the opposing pressure associated with the stretching PDMS walls. Note the nonlinear response arising from the cell wall (solid line), which contrasts with the classic example of a U-tube with a selectively permeable membrane barrier (dashed line). In both scenarios, water potential Ψ quantifies the energetic potential that leads to fluid flow. Like a chemical potential difference, water potential quantifies the free energy of a substance relative to a reference state. For water potential, the reference is pure water, in this case at atmospheric conditions. Water potential has units of pressure and is thus convenient to use for the current system as its mechanical contribution is simply the hydrostatic pressure (Pₜ for the PTAs, ρgh for the U-tube) and the presence of solute is captured via the osmotic potential Ψ_{Π}. The summation of these potentials for fluid flow lead to an expression for the water potential within the cavity,

Ψ_{PTA}=Ψ_{Π}+Pₜ, (Equation 1)

which, when combined with the potential of the surroundings, determines if fluid is driven into or out of a cavity. Here, Ψ_{Π}=−iCRT and i, C, R, and T and are the solute dissociation factor, solute concentration, universal gas constant, and temperature, respectively. Equilibrium occurs when the potential difference between the cells and the water bath vanishes, Ψ_{PTA}−Ψ_{bath}=0. Using a deionized water bath at atmospheric conditions, Ψ_{bath}=0 and, under the experimental conditions, Ψ_{PTA}=0 at equilibrium. (This equilibrium condition is the same as the chemical potential equilibrium.) FIG. 6A illustrates the balance of the components of this potential driving fluid flow by plotting −Ψ_{Π} and Pₜ versus the degree of swelling. For the initial salt concentration at zero deformation, Ψ_{PTA}<0 since Pₜ=0. As water moves into the cells (or cavities), the solution dilutes, making Ψ_{Π} less negative. The simultaneous increase in cell volume stretches the walls, increasing Pₜ. Equilibrium occurs at the intersection of the osmotic −Ψ_{Π} and turgor Pₜ curves. As FIG. 6B shows, a higher crosslink density yields a stiffer, less stretchable PDMS (see Table 1), which increases resistance of the cell wall to swelling. Lower initial salt concentration C₀ reduces the driving force for swelling (see left-most curve). The highest swelling occurs for cell walls with a lower crosslink density enclosing fluid-filled cells with a higher salt concentration (point D).

TABLE 1
Comparison of PDMS constitutive parameters

| | | | Swelling fit | | Composite estimate |
|---|---|---|---|---|---|
| | Tensile fit | | elastomer + | | elastomer + |
| Formulation | elastomer only | | nanoparticles | | nanoparticles |
| ratio | E_{PDMS} | | E_{w} | | \tilde{E}_{w} |
| (A:B) | (kPa) | Jₗᵢₘ | (kPa) | Jₗᵢₘ | (kPa) |
| 1:1 | 291.2 | 15.98 | 929.31 | 5.96 | 641 |
| 2:1 | 606.7 | 7.45 | 1,481.5 | 3.49 | 1,335 |
| 3:1 | 816.8 | 5.08 | 1,753.1 | 2.59 | 1,797 |

To validate this description of the anticipated behavior, osmotically-active closed-cell composites (PTAs) comprising three PDMS formulations of varying crosslink density are immersed in deionized water (see Table 2). Wall material formulations use differing ratios of a two-part (A & B) commercial silicone, Solaris™, and hexane. The fluid-filled cells have salt concentrations ranging from 0.01 to 0.2 m. (The unit m denotes molality, defined as the number of moles of solute per mass [in kilograms] of solvent.) As FIG. 6C shows, swelling throughout the time-dependent response, denoted as the ratio of the sample volume over the initial sample volume V/V₀, decreases with decreasing salt concentration. The time dependence is captured by an exponential saturation curve (V_{eq}/V₀[1−e−^{t/τ}]) (solid lines). At lower salt concentrations, an equilibrium value V_{eq}/V₀ is reached. For the highest salt concentrations, the swelling reaches a critical value before abruptly decreasing. This apparent deswelling is interpreted as the loss of water accompanying rupture of the thin cell walls due to excessive stretching, as visual observation suggests a loss of sample integrity. Such rupture resembles plasmoptysis in plants. Salt leakage appears to be minimal over the timescale of these experiments. These data also show that a larger initial salt concentration corresponds to a greater initial water potential difference, increasing the driving force for fluid influx. As a result, an increase in the initial slope is observed as a function of increasing concentration. Analogously, the initial rate of swelling is unaffected by the wall mechanical response when the initial salt concentration is the same, as shown in FIG. 6D. The latter swelling curves follow the same trajectory until the wall material's stiffness and finite stretchability take over the response, leading to equilibrium. As predicted in FIG. 6B, given the same initial salt concentration, PTAs with higher crosslinker density swell less.

TABLE 2
PTA fabrication parameters

| Formulation | | Mixing | Pre-fractionation | | Post-fractionation | |
|---|---|---|---|---|---|---|
| ratio | Hexane | speed | centrifugal force | | centrifugal force | |
| (A:B) | (wt %) | (rpm) | (×g) | | (×g) | |
| 1:1 | 20 | 600 | 3,000; 15 | min | 3,000; 30 | min |
| 2:1 | 20 | 600 | 2,900; 15 | min | 3,000; 40 | min |
| 3:1 | 23 | 700 | 2,400; 8 | min | 3,000; 20 | min |
| 1:1 (5% | 20 | 600 | 2,500; 10 | min | 3,000; 20 | min |
| glycerol) | | | | | | |
| 1:1 (33% | 20 | 600 | 1,300; 15 | min | 3,000; 7 | min |
| glycerol) | | | | | | |

A simple, quantitative model captures the equilibrium swelling for PTAs. It also enables the extraction of wall material properties, via fitting, which are critical when comparing stiffening observations with composite modulus theories. Inspired by previous treatments of plant tissue, the PTA composite structure is modeled as an inflating balloon. The spherical shell is filled with an incompressible fluid having an initial water potential dictated by the salt concentration. For an infinitely thin and stretchable shell wall, the salt concentration would be solely responsible for equilibrium swelling and equilibrium, Ψ_{PTA}−Ψ_{bath}=0, would be possible only when water influx is infinite. The magnitude of the turgor pressure Pₜ counteracting this limit is estimated by treating the wall material as incompressible and hyperelastic. The response of a single, cell-sized sphere is identical to a collection of non-interacting cell-sized spheres, which approximate the composite structure of the PTAs. Since the wall is incompressible, selectively permeable, and does not swell, an increase in volume decreases the osmotic potential Π according to ΠV=Π₀V₀, where Π₀ is the initial osmotic potential iC₀RT=−Ψ_{Π,0}. Simultaneously, Pₜ is given by the inflation of a nonlinear elastic balloon of finite wall thickness assuming a strain-stiffening, Gent material model. At lower stretches, the Gent material model recovers a neo-Hookean response having Young's modulus E. At larger stretches, the model strain stiffens due to a finite stretch limit governed by the limiting stretch parameter Jₗᵢₘ (in this geometry, Jₗᵢₘ=5 corresponds to infinite turgor pressure at V/V₀≈8). Thus, for a given PTA geometry, osmotic potential, and wall material, the model estimates the equilibrium expansion ratio, i.e., when Ψ_{PTA}=0.

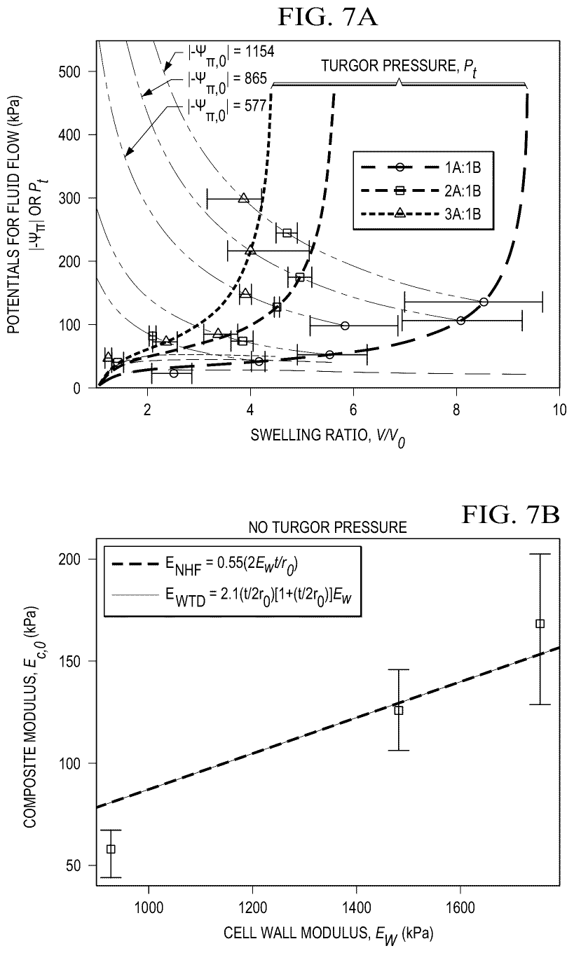

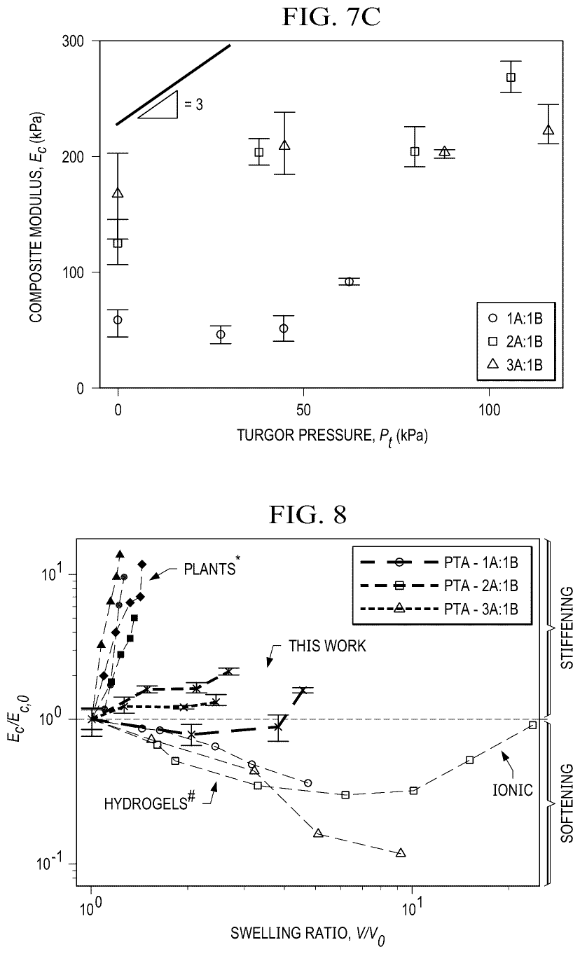

The equilibrium response of the PTAs is captured as illustrated by the solid curves in FIG. 7A using two fit parameters governing the wall material response, E_{w} and Jₗᵢₘ. The data (squares) correspond to the equilibrium volume expansion ratio of the PTAs as estimated from exponential fits of the time-dependent swelling data in FIGS. 6C and 6D. Dashed lines describe the decrease in osmotic potential of the samples as they swell. A neo-Hookean response (dotted lines) fails to capture the arrested swelling observed at higher osmotic potential. Rather, describing the PTA's equilibrium response requires both strain stiffening and large deformation behavior for the wall material.

The cell wall is composed of both PDMS and a high concentration of silica nanoparticles, which act as the emulsion stabilizer during sample fabrication. Thus, material properties obtained via the swelling model differ from those determined from fitting tensile test data on pure PDMS of the same formulation: (1) E_{PDMS} is higher, and (2) the limiting stretch (Jₗᵢₘ) is lower (e.g., see Table 1) for all three formulations. (PDMS with comparable nanoparticle concentrations cannot be fabricated due to handling and particle dispersion constraints.) Nanoparticles are well known for producing a stiffening effect on elastomers. At the high volume fraction at which they occur around the cells, the rigid nanoparticles may also decrease the overall stretchability of the PDMS plus nanoparticle composite walls. Employing the assumption that all nanoparticles added to the sample pre-centrifugation locate within the polymerized high internal phase emulsion (polyHIPE), an enhanced Young's modulus of the cell walls is calculated using Mooney's modified version of Einstein's equation to find an estimate of the Young's modulus of the particulate composite walls, \tilde{E}_{w} (see Table 1). The nearness of the swelling fit modulus to that estimated using Mooney's modification suggests that other effects, such as mechanical constraints due to inter-cavity interactions associated with the complex closed-cell structure yield only minor corrections to the fitted material parameters.

Swell Stiffening

Given their closely spaced fluid-filled cells separated by thin cell walls, PTAs may be described as fluid-filled foams. Like other foams, the Young's modulus of fluid-filled foams is less than the modulus of the cell wall material and is a function of the structure; e.g., the cell size r₀ and wall thickness t. Unlike typical foams, PTAs are filled with an incompressible fluid. The fluid incompressibility restricts wall deformation to primarily stretching, reducing or eliminating bending and buckling. We find that in the absence of turgor pressure (Pₜ=0), the modulus of the PTAs generally follows the previous predictions of both Nilsson et al. (“Nilsson”) and Warner et al. (“Warner”), which assume linear elastic cell walls of modulus E_{w} (see FIG. 7B). Using material constitutive parameters obtained from equilibrium swelling, the geometric fit constant for Nilsson's prediction falls between their predictions for a cubic geometry loaded diagonally and an isodiametric tetrakaidecahedral geometry with two square faces perpendicular to the direction of applied force. The cells in the PTAs are polyhedral, similar to plant parenchyma cells, suggesting they would be well represented by a tetrakaidecahedron. Also obtained is a constant of order one (2.1) for Warner's scaling theory, indicating reasonable agreement.

Unfortunately, there is no consensus as to the effect of turgor pressure on the constitutive response of an incompressible fluid-filled (not gas-filled) closed-cell foam. Even the theories by Nilsson and Warner vastly differ in their prediction of the turgor-pressure-dependent response. Nilsson finds that the fluid-filled foam composite modulus E_{c} is linearly proportional to turgor pressure with a constant of proportionality≈3. Warner's framework (the Warner, Theil, and Donald [WTD] framework) was later adopted to suggest that E_{c} is independent of turgor pressure for small swelling deformations. The latter appears less likely given the predictions of a more complex framework for ideal gas-filled, closed-cell elastomeric foams. That work predicts that increased pore pressure leads to an increase in stiffness.

As FIG. 7C shows, the modulus of the osmotically-active closed-cell composite is maintained or increases with Pₜ for all PDMS formulations. To plot this response, turgor pressure is first determined as a function of the equilibrium swelling. As established in the earlier swelling discussion, at equilibrium, water potential due to osmotic pressure is canceled out by the turgor pressure. Osmotic potential (Ψ_{Π}) is proportional to the total change in cavity volume,

Ψ_{Π}=Ψ_{Π}⁰V₀/V. (Equation 2)

Thus, at equilibrium, Pₜ is equal in magnitude to the initial osmotic potential Ψ_{Π}⁰ (given by Ψ_{π}⁰=−iC₀RT) divided by the volume expansion ratio at equilibrium V_{eq}/V₀. It is observed that the rate of modulus increase with respect to turgor pressure is greatest in all materials at the highest observable turgor pressures. This upturn for all three formulations corresponds to the onset of strain stiffening for the cell walls (the third point for each formulation in the equilibrium swelling curves shown in FIG. 7A). These results suggest nonlinear cell wall behavior plays a role in the stiffness response as well as the swelling response, as found in the previous section. The observation that a modulus increase coincides with strain stiffening is also replicated with a naive adaptation of the WTD framework. However, this approach may not be predictive. Briefly, this approach uses the WTD expression (see FIG. 7B) to predict E_{c} after modifying the inputs E_{w}, r₀, and t from the unstretched state by determining the instantaneous material modulus and geometric changes associated with an expanding thick spherical shell. Together, these findings motivate the need for a turgor-pressure-dependent constitutive model for closed-cell, incompressible fluid-filled elastomeric foams with high pore fraction.

As FIG. 8 demonstrates, PTAs comprising elastomeric cell walls occupy a unique niche in the design space for high-water-content materials exhibiting osmosis-driven deformation. The osmotically-active closed-cell composites exhibit two to four times the swelling-induced deformation capabilities observed in plant tissues. Their stiffness is less sensitive to swelling than plants, which may in part be due to a lack of the air pockets typically present in plant tissue, but is probably due to the opposing nonlinear response of plant cell walls compared with PTA walls. Plant cells strain stiffen at relatively low values of stretch (1.05-1.1, corresponding approximately to V/V₀˜1.16-1.3). At slightly higher swelling, the cell walls strain soften, further widening the gap illustrated in FIG. 8, before the onset of limiting stretch leads them to increase in stiffness. The magnitude of PTA swelling is similar to that typical of hydrogels, as FIG. 8 shows. However, as previously mentioned, hydrogels soften rather than stiffen upon swelling, making them unsuitable for actuation tasks requiring the application of or resistance to high forces. Like hydrogels, the PTAs described herein require only a water potential difference; the continuous phase polymer (a silicone in this work) may be readily exchanged to provide the selective permeability desired for a given application environment. Applications such as responsive valves and active biomaterials may benefit from such component interchangeability, as well as large, active deformations and a simultaneous ability to carry or resist load.

Actuation Proof-of-Concept

Plant tissues are well known for their ability to displace soil and rocks or lift themselves against gravity by curling or bending. The fact that osmotically-active closed-cell composites also stiffen upon swelling is exploited to mimic similar functionalities. Referring to FIG. 3, bilayer actuators 300 demonstrated here include the osmotically-active closed-cell composite (PTA) 100 attached to an object 200 (a PDMS layer in this example). The images of FIG. 5 illustrate how the bilayer actuator 300 curls due to differential swelling, a behavior that is unaffected by the addition of a 5-g weight (bottom-most image). In contrast, a hydrogel/PDMS actuator of similar initial stiffness cannot lift the additional weight (I), as shown in FIG. 6 (bottom-most image). The deformations of both actuators are comparable when unloaded. The hydrogel composition is chosen such that the swelling ratios after 72 h are the same. Additionally, the initial modulus of the osmotically-active closed-cell composite (E_{PTA}≈58 kPa) and hydrogel (E_{hydrogel}≈49 kPa) are very close, giving them similar early swelling force capabilities. During unweighted actuation, the stress within the actuating layer is ˜3 kPa in both bilayer actuators according to a modified version of Stoney's formula developed by Cai that is valid even when the gel layer is thicker than the substrate. In the loaded scenario, the hydrogel is incapable of supporting sufficient stress within the object, whereas an approximate maximum stress of at least 100 kPa is supported by the PTA according to small deformation composite beam theory. Thus, the osmotically-active closed-cell composite produces a force at least 10 times higher than the hydrogel. It is believed that much larger stresses could be accommodated given that the rate of actuation is virtually unaffected by the 5-g load. Referring again to FIG. 3, the bilayer actuator 300 operates when the osmotically-active closed-cell composite 100 swells relative to the object (PDMS layer) 200 that does not, bending the object 200 in a direction away from the composite 100.

Leveraging this bilayer actuation concept, PTA actuators may be molded and/or cut into shapes to yield specific motions, such as a soft gripper. In light of recent interest in hydrogels for biomedical soft robots, these results suggest that osmotically-active closed-cell composites may provide previously unachievable functionality for soft tissue therapies. Further, the active and high load capabilities of PTAs are particularly critical in light of recent understanding of the importance of mechanical environment on cell response.

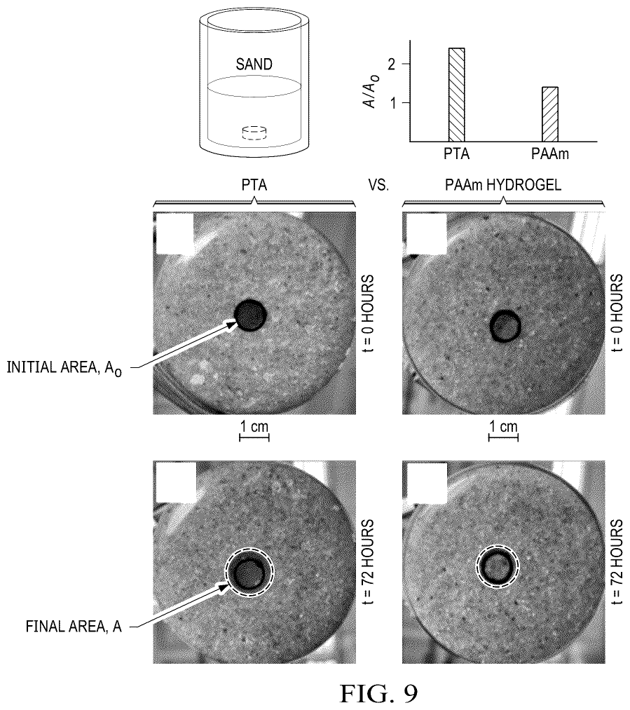

The PTAs can also operate within confined environments. To penetrate into the ground, plant roots need to apply forces high enough to dislodge soil or obstacles they encounter. Testing similar “pushing” capabilities of the osmotically-active closed-cell composite, 0.15-g discs are buried under 5 cm of sand, as shown in FIG. 9. (For faster [but temporary] swelling capacity, the PTA is fabricated with a 33% glycerol solution as the fluid within the fluid-filled cells. Although glycerol provides a larger initial driving force for fluid flow, it is not retained as well by the PDMS cell walls and thus leaks out after long periods. A PAAm hydrogel is made to have the same time-dependent unconstrained swelling as the PTA with 33% glycerol solution.) The sand is filled in with water, resulting in a net load of 300 g on the top of the disc, which simultaneously compacts the sand around it. The images labeled “L” and “M” show the discs from beneath the sand immediately upon burial. A black marker line on the bottom of the beaker indicates the initial disc area A₀. After 72 h, the PTA disc more than doubles in area A, as shown in the image labeled “N,” extending outside of the marker line (white dashed line). The PAAm hydrogel disc subjected to the same conditions reaches only 56% of the PTA's final area, as can be seen in the image labeled “◯” and the bar graph. It follows that the PTA is capable of applying a much larger force within the confined environment. A similar response is observed for a PTA disc confined within a Styrofoam capsule. The PTA disc breaks open the capsule upon swelling, while a PAAm hydrogel disc simply oozes through the holes in the mesh screen that enabled water flow.

As described above, plant-like, large-deformation, high-water-content, forceful, and osmotically-active closed-cell composites, or PTAs, have been fabricated and tested. While plants produce large deformations by virtue of growth coupled with osmotic driving forces, PTAs and hydrogels instead overcome an inability to grow through the use of soft polymer networks. Hydrogels soften as they enlarge, but swelling in both non-vascular plant tissue and osmotically-active closed-cell composites is characterized by an ability to apply and/or withstand forces due to their capacity for turgor pressure. The usage of these closed-cell composites as nastic actuators is demonstrated and their equilibrium response is modeled using a single, fluid-filled, thick-walled, spherical shell consisting of a nonlinear elastic, strain-stiffening solid. Results suggest that finite deformation plays a critical role in modulating the composite stiffness as a function of the turgor pressure within the cells.

### Experimental Examples

Fabrication of Osmotically-Active Closed-Cell Composites (or PTAs)

In the experiments described in this disclosure, PTAs are fabricated by thermosetting a water-in-PDMS high-internal-phase emulsion. Colored NaCl solution comprises the aqueous phase. The oil phase is PDMS (Solaris, Smooth On) plus hexane (H-3341, Fisher Scientific). Silica nanoparticles (Aerosil R 974, Evonik Industries) stabilize the emulsion. First, the oil phase and stabilizer (2.8 wtₒ% are mixed (1 min; 3,500 rpm; dual asymmetric centrifugal [DAC] mixer, FlackTek), then 23 wtₑ% of the aqueous phase is added manually and gently stirred, followed by DAC mixing (12 min). To increase droplet density, the emulsion is centrifuged using a two-step fractionation method. The centrifuged samples are cured at 70° C. for 30 min, then at room temperature for a day. Changes in PDMS pre-polymer ratio and inner phase viscosity can affect the final cavity size. Table 2 provides the parameters used to ensure that cell size distribution for the closed-cell composites fabricated in this study remains constant.

Hydrogel-PDMS Bilayer Actuator Fabrication

Acrylamide (AAm) (Sigma-Aldrich 01700) is dissolved in deionized water to form a solution of concentration 1.4 m. For every 25 g of water, 0.1 g of N,N′-methylene-bis(acrylamide) (Sigma-Aldrich 146072), 0.25 g of α-ketoglutaric acid (Sigma-Aldrich 75890), 50 μL of 3-(trimethoxysilyl) propyl methacrylate (TMSPMA; Sigma-Aldrich 440159), and 0.05 g of sodium dodecyl sulfate (Sigma-Aldrich L3771) are added as the crosslinker, UV initiator, coupling agent, and surfactant respectively. Food coloring is added to enhance visualization. The solution is poured into a 3D-printed acrylonitrile butadiene styrene mold to a height of 2 mm and UV cured (8 W, 365 nm; 2 cm from the lamp) for 1 h. Fifty microliters of trietoxy(vinyl)silane (TEOVS; Sigma-Aldrich 175560) is added to 10 g of PDMS (Sylgard 184, Dow Corning) precursor mixed in the standard 10:1 (base:curing agent) ratio. The mix is degassed and poured (1-mm thickness) on the cured hydrogel. The PDMS is cured at 70° C. for 8 h followed by 16 h of room temperature curing. A rectangular bilayer sample (8 cm×1 cm) is cut using a blade.

PTA-PDMS Bilayer Actuator Fabrication

In this example, a PTA is fabricated with a 5% glycerol solution for the fluid-filled cells phase and a 1:1 PDMS (Solaris) continuous phase for the cell walls (see Table 2); generally speaking, it is preferred to use an osmolyte that does not leak through the cell walls. The centrifuged PTA is poured into a polyacrylic mold (8 cm×1 cm×3 mm) up to a height of 2 mm. PDMS precursor (Sylgard 184, Dow Corning) mixed in a 10:1 ratio is poured on top of the PTA layer to a thickness of 1 mm. The bilayer is cured at room temperature for 24 h and then extracted from the mold.

The subject matter of this disclosure may also relate to the following aspects:

A first aspect relates to an osmotically-active closed-cell composite comprising: a closed-cell structure including: fluid-filled cells comprising water and a solute; and cell walls separating the fluid-filled cells, the cell walls comprising a polymer permeable to water and impermeable to the solute, wherein the closed-cell structure is configured to undergo osmotically-induced swelling during exposure to an aqueous environment having a different chemical potential from the fluid-filled cells.

A second aspect relates to the osmotically-active closed-cell composite of the preceding aspect, wherein a swelling ratio V/V₀ of the closed-cell structure depends on: thickness of the cell walls, size of the fluid-filled cells, concentration of the solute in the water, constitutive response of the polymer, and/or chemical composition of the cell walls.

A third aspect relates to the osmotically-active closed-cell composite of any preceding aspect, wherein an elastic modulus E_{c} of the closed-cell structure remains substantially constant or increases with the osmotically-induced swelling.

A fourth aspect relates to the osmotically-active closed-cell composite of any preceding aspect, wherein the fluid-filled cells are polyhedral or curved in shape.

A fifth aspect relates to the osmotically-active closed-cell composite of any preceding aspect, wherein the fluid-filled cells account for at least about 74 vol. %, or at least about 80 vol. % of the closed-cell structure.

A sixth aspect relates to the osmotically-active closed-cell composite of any preceding aspect, wherein the closed-cell structure does not include glycerol.

A seventh aspect relates to the osmotically-active closed-cell composite of any preceding aspect, wherein the fluid-filled cells comprise a cell size r₀ of: at least about 10 microns, at least about 25 microns, or at least about 50 microns, and/or no greater than about 500 microns, no greater than about 250 microns, or no greater than 100 microns.

An eighth aspect relates to the osmotically-active closed-cell composite of any preceding aspect, wherein the cell walls have a thickness t of: at least about 0.1 micron, or at least about 1 micron, and/or up to about 10 microns.

A ninth aspect relates to the osmotically-active closed-cell composite of any preceding aspect, wherein the cell walls further comprise silica nanoparticles

A tenth aspect relates to the osmotically-active closed-cell composite of any preceding aspect, wherein the polymer comprises a homopolymer or a copolymer.

An eleventh aspect relates to the osmotically-active closed-cell composite of any preceding claim, wherein the polymer comprises an elastomer.

A twelfth aspect relates to the osmotically-active closed-cell composite of the preceding aspect, wherein the elastomer comprises silicone, natural rubber, polybutadiene, styrene butadiene rubber, polyurethane, polyisoprene, and/or neoprene.

A thirteenth aspect relates to the osmotically-active closed-cell composite of any preceding aspect, wherein the solute comprises an organic osmolyte.

A fourteenth aspect relates to the osmotically-active closed-cell composite of the preceding aspect, wherein the osmolyte comprises a protein, an amino acid, a polyol, a sugar, a polysaccharide such as alginate, a methylamine, and/or urea.

A fifteenth aspect relates to the osmotically-active closed-cell composite of any preceding aspect, wherein the solute comprises an ionic compound.

A sixteenth aspect relates to the osmotically-active closed-cell composite of any preceding aspect, wherein the ionic compound comprises a salt selected from the group consisting of sodium chloride, potassium chloride, sodium phosphate, sodium bicarbonate, ammonium sulfate, sodium sulfate, and ammonium acetate.

A seventeenth aspect relates to the osmotically-active closed-cell composite of any preceding aspect, wherein the solute has a concentration in the water in a range from greater than zero to a saturation concentration.

An eighteenth aspect relates to the osmotically-active closed-cell composite of the preceding aspect, wherein the solute comprises sodium chloride, and wherein a concentration of the solute is in the range from about 0.01 m to about 0.2 m, wherein m denotes molality, the number of moles of the solute per mass (in kg) of the water.

A nineteenth aspect relates to the osmotically-active closed-cell composite of any preceding aspect, wherein the solute comprises a gel.

A twentieth aspect relates to the osmotically-active closed-cell composite of the preceding aspect, wherein the solute comprises hydrogel beads or particles.

A twenty-first aspect relates to an osmotically-activated actuator comprising: the osmotically-active closed-cell composite of any preceding claim in contact with, attached to, or integrally formed with an object to be actuated via a swelling force.

A twenty-second aspect relates to the osmotically-activated actuator of the preceding aspect, wherein the object comprises an inanimate object, human tissue, and/or animal tissue.

A twenty-third aspect relates to the osmotically-activated actuator of any preceding aspect used for biomedical or industrial applications.

A twenty-fourth aspect relates to the osmotically-activated actuator of any preceding aspect, wherein the osmotically-active closed-cell composite is attached to the object by an adhesive bond.

A twenty-fifth aspect relates to the osmotically-activated actuator of any preceding aspect, wherein the osmotically-active closed-cell composite is integrally formed with the object during a molding or printing process.

A twenty-sixth aspect relates to an actuation method comprising: providing an actuator comprising an osmotically-active closed-cell composite in contact with an object to be actuated, the osmotically-active closed-cell composite comprising: a closed-cell structure including fluid-filled cells separated by cell walls, the fluid-filled cells comprising water and a solute, the cell walls comprising a polymer permeable to water and impermeable to the solute; exposing the actuator to an aqueous environment having a chemical potential different from a chemical potential of the fluid-filled cells of the closed-cell structure, whereby water from the aqueous environment diffuses through the cell walls and enters the fluid-filled cells, the closed-cell structure undergoing osmotically-induced swelling, thereby applying a swelling force to and effecting actuation of the object.

A twenty-seventh aspect relates to the actuation method of the preceding aspect, wherein the actuation continues until the chemical potential of the fluid-filled cells is the same as the chemical potential of the aqueous environment.

A twenty-eighth aspect relates to the actuation method of any preceding aspect, wherein the osmotically-active closed-cell composite is attached to or integrally formed with the object.

A twenty-ninth aspect relates to the actuation method of the preceding aspect, wherein the object to be actuated is attached to the osmotically-active closed-cell composite by an adhesive bond.

A thirtieth aspect relates to the actuation method of the twenty-eighth aspect, wherein the object to be actuated is integrally formed with the osmotically-active closed-cell composite during a molding process.

A thirty-first aspect relates to the actuation method of any preceding aspect, wherein the aqueous environment comprises liquid water and/or water vapor.

A thirty-second aspect relates to the actuation method of any preceding aspect, wherein exposing the actuator to the aqueous environment comprises: submerging the actuator in the liquid water and/or water vapor; positioning the actuator in a flow path of the liquid water and/or water vapor; and/or spraying the actuator with the liquid water and/or water vapor.

A thirty-third aspect relates to the actuation method of any preceding aspect, wherein an elastic modulus E_{c} of the closed-cell structure remains substantially constant or increases as the closed-cell structure undergoes osmotically-induced swelling.

A thirty-fourth aspect relates to the actuation method of any preceding aspect, wherein a final swelling ratio V/V₀ of the closed-cell structure depends on: thickness of the cell walls, size of the fluid-filled cells, concentration of the solute in the water, constitutive response of the polymer, and/or chemical composition of the cell walls.

A thirty-fifth aspect relates to the actuation method of any preceding aspect, wherein the object comprises an inanimate object, human tissue, and/or animal tissue.

A thirty-sixth aspect relates to the actuation method of any preceding aspect used for biomedical or industrial applications.

A thirty-seventh aspect relates to the actuation method of any preceding aspect, wherein the permeability of the polymer to the water is at least three orders of magnitude higher than permeability of the polymer to the solute.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein.

Furthermore, the advantages described above are not necessarily the only advantages of the invention, and it is not necessarily expected that all of the described advantages will be achieved with every embodiment of the invention.

1. An osmotically-active closed-cell composite comprising:
a closed-cell structure including:
fluid-filled cells comprising water and a solute; and
cell walls separating the fluid-filled cells, the cell walls comprising a polymer permeable to water and impermeable to the solute,
wherein the closed-cell structure is configured to undergo osmotically-induced swelling during exposure to an aqueous environment having a different chemical potential from the fluid-filled cells.

2. The osmotically-active closed-cell composite of claim 1, wherein an elastic modulus E_{c} of the closed-cell structure remains substantially constant or increases with the osmotically-induced swelling.

3. The osmotically-active closed-cell composite of claim 1, wherein the fluid-filled cells account for at least about 74 vol. % of the closed-cell structure.

4. The osmotically-active closed-cell composite of claim 1, wherein the closed-cell structure does not include glycerol.

5. The osmotically-active closed-cell composite of claim 1, wherein the cell walls further comprise silica nanoparticles.

6. The osmotically-active closed-cell composite of claim 1, wherein the polymer comprises an elastomer.

7. The osmotically-active closed-cell composite of claim 1, wherein the solute comprises an organic osmolyte.

8. The osmotically-active closed-cell composite of claim 7, wherein the osmolyte comprises a protein, an amino acid, a polyol, a sugar, a polysaccharide, alginate, a methylamine, and/or urea.

9. The osmotically-active closed-cell composite of claim 1, wherein the solute comprises an ionic compound.

10. The osmotically-active closed-cell composite of claim 1, wherein the ionic compound comprises a salt selected from the group consisting of sodium chloride, potassium chloride, sodium phosphate, sodium bicarbonate, ammonium sulfate, sodium sulfate, and ammonium acetate.

11. The osmotically-active closed-cell composite of claim 1, wherein the solute has a concentration in the water in a range from greater than zero to a saturation concentration.

12. The osmotically-active closed-cell composite of claim 1, wherein the solute comprises a gel.

13. An osmotically-activated actuator comprising:
the osmotically-active closed-cell composite of claim 1 in contact with, attached to, or integrally formed with an object to be actuated via a swelling force.

14. An actuation method comprising:
providing an actuator comprising an osmotically-active closed-cell composite in contact with an object to be actuated, the osmotically-active closed-cell composite comprising:
a closed-cell structure including fluid-filled cells separated by cell walls, the fluid-filled cells comprising water and a solute, the cell walls comprising a polymer permeable to water and impermeable to the solute;
exposing the actuator to an aqueous environment having a chemical potential different from a chemical potential of the fluid-filled cells of the closed-cell structure,
whereby water from the aqueous environment diffuses through the cell walls and enters the fluid-filled cells, the closed-cell structure undergoing osmotically-induced swelling,
thereby applying a swelling force to and effecting actuation of the object.

15. The method of claim 14, wherein the actuation continues until the chemical potential of the fluid-filled cells is the same as the chemical potential of the aqueous environment.

16. The method of claim 14, wherein the osmotically-active closed-cell composite is attached to or integrally formed with the object.

17. The actuation method of claim 14, wherein exposing the actuator to the aqueous environment comprises:
submerging the actuator in liquid water and/or water vapor;
positioning the actuator in a flow path of liquid water and/or water vapor; and/or
spraying the actuator with liquid water and/or water vapor.

18. The actuation method of claim 14, wherein an elastic modulus E_{c} of the closed-cell structure remains substantially constant or increases as the closed-cell structure undergoes osmotically-induced swelling.

19. The actuation method of claim 14, wherein the object comprises an inanimate object, human tissue, and/or animal tissue.

20. The actuation method of claim 14, wherein the permeability of the polymer to the water is at least three orders of magnitude higher than permeability of the polymer to the solute.
